(19) United States
(12) Patent Application Publication
(10) Pub. No.: US 2020/0390702 A1
(43) Pub. Date: Dec. 17, 2020

(54) LIPOSOMAL TAXANES FOR TREATMENT OF SCLC

(71) Applicant: Plus Therapeutics, Inc., San Diego, CA (US)

(72) Inventor: Mark T. Marino, Carlsbad, CA (US)

(21) Appl. No.: 16/637,468

(22) PCT Filed: Aug. 6, 2018
(86) PCT No.: PCT/US2018/045339

§ 371 Date: Feb. 7, 2020

Related U.S. Application Data
(60) Provisional application No. 62/542,993, filed on Aug. 9, 2017.

(51) Int. Cl. A61K 9/127; A61K 31/337

(52) U.S. Cl. CPC A61K 9/1271; A61K 31/337

(57) ABSTRACT

Methods for therapy for a small cell lung cancer (SCLC), which may or may not respond to a first-line therapy or that progresses following cessation of a first-line chemotherapy is provided. Some methods include the administration of composition that comprises a protein-stabilized form of docetaxel, optionally in conjunction with a regimen of supportive care.

### CROSS-REFERENCE TO RELATED APPLICATIONS

This application is a U.S. National Phase Application of PCT International Application Number PCT/US2018/045339, filed on Aug. 6, 2018, designating the United States of America and published in the English language, which is an International Application of and claims the benefit of priority to U.S. Provisional Application No. 62/542993, filed on Aug. 9, 2017. The disclosures of the above-referenced applications are hereby expressly incorporated by reference in their entireties.

### FIELD OF THE INVENTION

Some embodiments of the methods and compositions provided herein relate to the treatment or inhibition of a cancer including small cell lung cancer (SCLC), which may or may not respond to a first-line therapy or that progresses following cessation of a first-line chemotherapy.

### BACKGROUND

According to the Centers for Disease Control and American Cancer Society, more people in the U.S. die from lung cancer than any other type of cancer. For lung and bronchus cancer, it is estimated that there are 222,500 new cases and 155,870 deaths each year. Small cell lung cancer, or SCLC accounts for approximately 15% of bronchogenic carcinomas. SCLC cells are histologically distinguishable by being poorly differentiated, having a high mitotic index, displaying neuroendocrine markers such as calcitonin gene related polypeptide (CGRP) and neural cell adhesion molecule (NCAM1). SCLC is a fast-growing cancer that spreads rapidly and, for first-line treatment purposes, most physicians use a 2-stage system (see Table 1).

TABLE 1

| | | | Median | |
|---|---|---|---|---|
| | | | Survival | |
| SCLC | Standard of | Treatment | Time | Survival |
| Stage | Care | Goal | (Months)⁷ | Rate |
| Limited | Chemotherapy | Cure | 15-20 | ≤15% |
| | (etoposide + | | | at 5 years |
| | cisplatin or | | | |
| | carboplatin); | | | |
| | Radiotherapy | | | |
| Extensive | Chemotherapy | Symptom relief, | 9.4-12.8 | 5.2-19.5% |
| | (etoposide + | maintain | | at 2 years |
| | cisplatin or | quality of | | |
| | carboplatin) | life, prolong | | |
| | | survival | | |

All patients with extensive-stage disease and most patients with limited stage disease relapse within months of completing initial therapy.

Combination chemotherapy is currently considered standard first-line therapy for SCLC. The most common regimens include platinum (Pt) drugs such as cisplatin or carboplatin and etoposide. Unfortunately, despite the 40-90% response rate to first-line chemotherapy, long-term survival is unusual because patients develop resistance to chemotherapy and relapse. Without further, treatment, the overall expected mean survival after disease relapse is two to four months. Topotecan, a topoisomerase inhibitor I, is the only U.S. FDA drug approved for SCLC sensitive disease after failure of first-line therapy. Topotecan is administered as 1.5 mg/m² by intravenous infusion over 30 minutes daily for 5 consecutive days, starting on day 1 of a 21-day course, which is a significant burden for the SCLC patient. In a review of 6 phase II-III studies in which 631 relapsed cases were treated with intravenous topotecan, there was a 20.4% and 4.0% response rate in chemotherapy-sensitive and -refractory cases, respectively, which are disappointing in terms of risk-benefit.

There exists a need for therapies that improve cure rates in patients with Limited Stage disease and extend survival of patients with Extensive Stage disease over 1 year. There is also a need for therapeutics with better safety profiles both for patients who cannot tolerate the adverse effects of first-line chemo-radiotherapy and for relapsed/refractory patients who receive the only FDA approved therapy, topotecan, which has an unfavorable toxicity profile.

### SUMMARY

The methods and compositions provided herein provide for novel means to treat or inhibit neoplastic diseases, including but not limited to lung cancers, including small cell lung cancer. Accordingly, some methods provide for treating, inhibiting, or ameliorating a cancer, which includes identifying a subject with a cancer, and administering the subject a therapeutically effective amount of a protein-stabilized liposome that comprises docetaxel. Preferably, the cancer is a lung cancer, and more preferably the cancer is small cell lung cancer (SCLC). In particular, some embodiments provide methods of treating a subject that has refractory SCLC; for example the subject may be been responsive to an initial therapy (e.g., platinum-based therapy, or the like), or the subject may have responded to an initial therapy (e.g., a platinum-based therapy, or the like), but relapsed within three months or 45 days of cessation of the initial therapy. In some embodiments, the response to initial therapy can be a partial response, whereas in some embodiments, the response to initial therapy can be a complete response. In some embodiments, the subject has sensitive SCLC. In some embodiments, the subject has limited stage SCLC. In some embodiments, the subject has extensive stage SCLC. In some embodiments, the subject is administered an amount of a protein-stabilized liposome that comprises docetaxel (PSL doceteaxel) that is sufficient to extend progression-free survival of the subject. In some embodiments, the subject is administered an amount of PSL docetaxel sufficient to extend the overall life expectancy of the subject. In some embodiments, the amount provides between about 50 to 120 mg/m² docetaxel, and preferably between 75-90 mg/m² docetaxel. In some embodiments, the PSL docetaxel includes albumin or an albumin derivative or variant (e.g., a mutant albumin). In some embodiments, the PSL docetaxel is a stealth liposome. In some embodiments, the PSL docetaxel includes methoxypolyethylene glycol (MPEG). In some embodiments, the protein-stabilized liposome includes 1,2-distearoyl-sn-glycero-3-phosphoethanolamine-N-[biotinyl(polyethylene glycol)-2000] (PEG-DSPE).

Other embodiments provide a method of improving overall survival in a subject that has refractory SCLC, by identifying a subject that has refractory SCLS and administering to the subject a therapeutically effective amount of a protein-stabilized liposome that includes docetaxel. The subject may have had no response to an initial therapy (e.g., a platinum-based therapy or the like), or the subject may have had an initial response, but relapsed within 3 months (e.g., 45 days or less) after the initial therapy. In some embodiments, the therapeutically effective amount provides between about 50 to 120 mg/m² docetaxel, and preferably between 75-90 mg/m² docetaxel. In some embodiments, the protein-stabilized liposome includes albumin or an albumin derivative or variant (e.g., a mutant albumin). In some embodiments, the protein-stabilized liposome is a stealth liposome. For example, in some embodiments, the protein-stabilized liposome includes methoxypolyethylene glycol. In some embodiments, the protein-stabilized liposome includes 1,2-distearoyl-sn-glycero-3-phosphoethanolamine-N-[biotinyl(polyethylene glycol)-2000] (PEG-DSPE).

Still other embodiments provide a method of improving overall survival in a subject that has refractory SCLC, including identifying a subject that has refractory SCLC, and administering to the subject a therapeutically effective amount of a protein-stabilized liposome that comprises docetaxel. In some embodiments, the subject had no response to an initial therapy (e.g., a platinum-based therapy). In some embodiments, the subject had a response to an initial therapy (e.g., a platinum-based therapy), but progressed or relapsed in less than three months (e.g., 45 days or less). In some embodiments, the subject has limited stage SCLC. In some embodiments, the subject has extensive-stage SCLC. In some embodiments, the therapeutically effective amount provides between about 50 to 120 mg/m² docetaxel, and preferably between 75-90 mg/m² docetaxel. In some embodiments, the protein-stabilized liposome includes albumin. In some embodiments, the protein-stabilized liposome is a stealth liposome. For example, in some embodiments, the protein-stabilized liposome includes methoxypolyethylene glycol. In some embodiments, the protein-stabilized liposome includes 1,2-distearoyl-sn-glycero-3-phosphoethanolamine-N-[biotinyl(polyethylene glycol)-2000] (PEG-DSPE).

Some embodiments provide for use of protein-stabilized liposomes as disclosed herein, for treating SCLC (e.g., refractory SCLC or sensitive SCLC). In some embodiments, the protein-stabilized liposomes are for use in the treatment or a therapy for subjects that were non-responsive to an initial therapy (e.g., a platinum-based therapy), or that were responsive to an initial therapy (e.g., partially or fully responsive to a platinum based therapy or other therapy). In accordance with some embodiments, provided herein are protein-stabilized liposomes for use in the treatment of either limited stage or end stage SCLC, to extend progression-free survival of the subject, and/or to extend overall life expectancy of the subject. In some embodiments, the protein-stabilized liposome includes albumin or an albumin derivative or variant (e.g., a mutant albumin). In some embodiments, the protein-stabilized liposome includes methoxypolyethylene glycol. In some embodiments, the protein-stabilized liposome is formulated to provide between about 50-120 mg/m² docetaxel to the subject; and more preferably between about 75-90 mg/m² docetaxel. In some embodiments, the protein-stabilized liposome includes albumin. In some embodiments, the protein-stabilized liposome is a stealth liposome. For example, in some embodiments, the protein-stabilized liposome includes methoxypolyethylene glycol. In some embodiments, the protein-stabilized liposome includes 1,2-distearoyl-sn-glycero-3-phosphoethanolamine-N-[biotinyl(polyethylene glycol)-2000] (PEG-DSPE).

Some embodiments provide for use of protein-stabilized liposomes as described herein for improving overall survival in a subject that has refractory SCLC. In some embodiments, the protein-stabilized liposomes are for use in subjects that had no response to an initial therapy for SCLC (e.g., a platinum based therapy or the like). In some embodiments, the protein-stabilized liposomes are for use in subject that had a response (either full or partial), to an initial therapy for SCLC (e.g., a platinum based therapy or the like), but that relapsed in less than three months. In some embodiments, the protein-stabilized liposomes are for use in subjects with limited stage SCLC. In some embodiments, the protein-stabilized liposomes are for use in subjects with extensive stage SCLC. In some embodiments, the protein-stabilized liposome is formulated to provide between about 50-120 mg/m² docetaxel to the subject; and more preferably between about 75-90 mg/m² docetaxel. In some embodiments, the protein-stabilized liposome includes albumin. In some embodiments, the protein-stabilized liposome is a stealth liposome. For example, in some embodiments, the protein-stabilized liposome includes methoxypolyethylene glycol. In some embodiments, the protein-stabilized liposome includes 1,2-distearoyl-sn-glycero-3-phosphoethanolamine-N-[biotinyl(polyethylene glycol)-2000] (PEG-DSPE).

Some embodiments provide for use of protein-stabilized liposomes as described herein for improving progression-free survival in a subject that has refractory SCLC. In some embodiments, the protein-stabilized liposomes are for use in subjects that had no response to an initial therapy for SCLC (e.g., a platinum based therapy or the like). In some embodiments, the protein-stabilized liposomes are for use in subject that had a response (either full or partial), to an initial therapy for SCLC (e.g., a platinum based therapy or the like), but that relapsed in less than three months. In some embodiments, the protein-stabilized liposomes are for use in subjects with limited stage SCLC. In some embodiments, the protein-stabilized liposomes are for use in subjects with extensive stage SCLC. In some embodiments, the protein-stabilized liposome is formulated to provide between about 50-120 mg/m² docetaxel to the subject; and more preferably between about 75-90 mg/m² docetaxel. In some embodiments, the protein-stabilized liposome includes albumin. In some embodiments, the protein-stabilized liposome is a stealth liposome. For example, in some embodiments, the protein-stabilized liposome includes methoxypolyethylene glycol. In some embodiments, the protein-stabilized liposome includes 1,2-distearoyl-sn-glycero-3-phosphoethanolamine-N-[biotinyl(polyethylene glycol)-2000] (PEG-DSPE).

Some embodiments provide kits for treating, inhibiting, or ameliorating SCLC, and/or for improving overall survival in a subject with refractory SCLC, and/or for improving progression-free survival in a subject that has refractory SCLC. In some embodiments, the kit includes a composition comprising a protein-stabilized liposome that includes docetaxel. In some embodiments, the protein-stabilized liposome is lyophilized. In some embodiments, the kit can include a reagent for reconstitution of lyophilized protein-stabilized liposomes. In some embodiments, the protein-stabilized liposome includes albumin or an albumin derivative or variant (e.g., a mutant albumin). In some embodiments, the protein-stabilized liposome is a stealth liposome. For example, in some embodiments, the protein-stabilized liposome includes methoxypolyethylene glycol. In some embodiments, the protein-stabilized liposome includes 1,2-distearoyl-sn-glycero-3-phosphoethanolamine-N-[biotinyl(polyethylene glycol)-2000] (PEG-DSPE).

### BRIEF DESCRIPTION OF THE DRAWINGS

FIG. 1 is a graph showing the concentration (ng/ml) of free docetaxel (diamonds) and PLS docextael (squares) over time in a minipig model, as described in Example 1.

FIG. 2 is a bar graph showing the AUC [uM*hr/mg] in serum for free docetaxel and PSL docetaxel in a minipig model, as described in Example 1.

FIG. 3 is a graph showing the concentration [ng/m1] of docetaxel in the lungs at various timepoints after administration in mice that were administered free docetaxel (diamonds) or PSL docetaxel (squares).

### DETAILED DESCRIPTION

### Methods of Therapy

The present invention provides a method for treating, inhibiting, or ameliorating small cell lung cancer (SCLC) comprising: administering a protein-stabilized liposomal docetaxel (PSL docetaxel) formulation (or composition comprising PSL docetaxel) to a subject that has SCLC.

In some embodiments, the PSL docetaxel is administered as a first-line therapy for SCLC. Accordingly, provided are methods wherein a subject is identified as having SCLC, and the subject is administered PSL docetaxel.

In some embodiments, the PSL docetaxel is administered as a second line therapy, i.e., is administered to a subject that received an initial therapy for SCLC. In some embodiments, the subject has “refractory” SCLC. Refractory SCLC refers to SCLC that is non-responsive to a first-line therapy (stable or progressive through first-line therapy) or that has responded to first-line therapy, but then has relapsed/progressed within 90 days after cessation (i.e., after the last dose) of first-line therapy. In some embodiments, the subject has “early relapsing” SCLC. Early relapsing SCLC refers to SCLC that initially responded to first line therapy, but relapsed/progressed within 45 days (1.5 mos.) after the end of said therapy. For example, in some embodiments, the subject responded to first line therapy but relapsed or progressed in 90 days, 85 days, 80 days, 75 days, 70 days, 65 days, 60 days, 55 days, 50 days, 45 days, 40 days, 35 days, 30 days, 25 days, 20 days, 15 days, 10 days, 5 days, or less (but not zero), or within a range defined by any two of the aforementioned time points. “Non-responsive” subjects refer to subjects that fail to respond in that the SCLC remains stable or progresses through or during initial or “first-line” therapy. Non responsive SCLC includes SCLC does not respond to first-line treatment in that the patient's SCLC remains stable during first-line treatment of at least three cycles (treatment intervals), and until second line treatment (e.g. PSL docetaxel), or that progresses during first-line treatment, including SCLC that progresses throughout first-line treatment of at least two cycles (treatment sessions), and continues to progress until initiation of second line treatment (e.g., PSL docetaxel). Accordingly, provided are methods wherein a subject is identified as having refractory SCLC (e.g., early-relapsing SCLC, non-responsive SCLC, or SCLC that has relapsed/progressed within 90 days after the last dose of first line therapy), and administered PSL docetaxel.

In some embodiments, the subject has “sensitive SCLC”. Subjects with “sensitive SCLC” are those that have maintained a response to initial treatment for three months or longer. Patients who initially respond, but then relapse within 90-180 days after the cessation of first-line therapy, are considered herein to have SCLC that is “90-180 progressive” or to have SCLC that is believed to be more sensitive to first-line therapy. Accordingly, some embodiments provide methods of treating, inhibiting, or ameliorating 90-180 day progressive SCLC, wherein a subject is identified as having 90-180 day progressive SCLC, and is administering PSL docetaxel. Some embodiments provide methods of treating, inhibiting, or ameliorating sensitive SCLC, wherein a subject with sensitive SCLC is identified, and administered PSL docetaxel as described herein.

In some embodiments, the subject's response to initial therapy was partial. In some embodiments, the subject's response to initial therapy was complete. As used herein, the term “partial response” or “partial remission” refers to at least a 50% reduction in measurable tumor. In some embodiments, the patient is assessed using the Response Evaluation Criteria in Solid Tumors (RECIST). As used herein the term “complete response” or complete “remission” refers to disappearance of all signs of cancer in response to treatment. Accordingly, some embodiments provide methods of treating SCLC comprising identifying a subject that had a partial response to an initial therapy, and administering PSL docetaxel to the subject. Some embodiments provide methods of treating SCLC comprising identifying a subject that had a complete response to SCLC, and administering to the subject PSL docetaxel.

In some embodiments, the subject has limited stage SCLC. “Limited stage SCLC” refers to SCLC that has only on one side of the chest and that can be treated with a single radiation field. This generally includes cancers that are only in one lung (unless tumors are widespread throughout the lung), and that may have also reached the lymph nodes (e.g., supraventricular nodes or mediastinal lymph nodes) on the same side of the chest

In some embodiments, the subject has extensive stage SCLC. “Extensive stage SCLC” refers to SCLC that has spread widely throughout the lung, to the other lung, to lymph nodes on the other side of the chest, or to other parts of the body (e.g., bone marrow). Extensive stage SCLC can refer to SCLC that has spread to the fluid around the lung. The present method can also be effective to treat SCLC that has metastasized to remote sites, such as to the brain, in the bone marrow, liver, or the like.

In the embodiments wherein PSL docetaxel is administered as a second-line therapy, the initial therapy can be a platinum-containing anti-cancer agent (e.g., carboplatin, cisplatin, picoplatin, and/or the like), a non-platinum-containing agents (e.g., CAV (cyclophosphamide/adriamycin/vincrisinte), etoposide and/or irinotecan, or the like). In some embodiments, the initial therapy is a targeted therapy, or antibody therapy. In some embodiments, the initial therapy is radiation therapy. In some embodiments, the initial therapy is radiation therapy in combination with one or more of a platinum-containing anti-cancer agent, a non-platinum containing anti-cancer agent, and/or targeted therapy.

In some embodiments of the methods disclosed herein wherein the subject is administered PSL docetaxel as a second line therapy, the subject does not require or receive third-line chemotherapy, following progression of the SCLC, for a period of time, e.g., for up to about one year from progression of the SCLC, or for at least up to about 60 days or 60days from progression of the SCLC.

In some embodiments, the subject is administered a therapeutically effective amount of PSL docetaxel. A “therapeutically effective amount” refers to an amount that is sufficient to inhibit, delay, or reduce the growth of a SCLC tumor. For example, in some embodiments, the therapeutically effective amount is sufficient to stop the growth of an SCLC tumor. In some embodiments, the therapeutically effective amount is sufficient to reduce the tumor size of an SCLC tumor by at least 5%, at least 10% at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more or within a range defined by any two of the aforementioned amounts. In some embodiments, a therapeutically effective amount refers to an amount of PSL that is sufficient to extend progression-free survival of the subject. For example in some embodiments, the therapeutically effective amount is sufficient to extend progression-free survival by 2 days, 5 days, 1 week, 2 weeks, 3 weeks, 4 weeks, 5 weeks, 6 weeks, 7 weeks, 8 weeks, 9 weeks, 10 weeks, 12 weeks, 1 month, 2 months, 3 months, 4 months 5 months, 6 months, 7 months, 8 months, 9 months 10 months, 11 months, a year, or more, or within a range of time defined by any two of the aforementioned time points. In some embodiments, the therapeutically effective amount is sufficient to improve the overall survival of the subject, e.g., by 2 days, 5 days, 1 week, 2 weeks, 3 weeks, 4 weeks, 5 weeks, 6 weeks, 7 weeks, 8 weeks, 9 weeks, 10 weeks, 12 weeks, 1 month, 2 months, 3 months, 4 months 5 months, 6 months, 7 months, 8 months, 9 months 10 months, 11 months, a year, or more, or within a range of time defined by any two of the aforementioned time points.

The methods disclosed herein can result in control of the SCLC and can extend the life (e.g., the median survival time or MST) in non-responsive or early-relapsing patients compared to the life of such SCLC patients receiving only a regimen of best supportive care (BSC) after failure of first-line therapy, either without the administration of subsequent, adjunct chemotherapy (third-line therapy) to either patient group, or if both patient groups receive subsequent adjunct chemotherapy (and the results are balanced for receipt of third-line therapy).

In some embodiments, PSL docetaxel second-line therapy is administered for patients who do not receive third-line therapy, either due to patient choice or for whom third-line therapy is contraindicated or otherwise not employed or not an option. For patients in the PSL docetaxel treatment group who are no more than, or that are preferably less than, 50 years of age, and/or who exhibit an ECOG performance status of 0 or 1 prior to initiation of treatment(s), the present PSL docetaxel treatment(s) are particularly beneficial.

In some embodiments, the methods described herein include administration of best supportive care, in addition to PSL docetaxel. “Best supportive care” (BSC) for SCLC includes a number of palliative treatments that may also have limited therapeutic efficacy against lung cancer, but are not considered to be curative. For example, in one embodiment, BSC includes one or more, and preferably all, of irradiation to control symptoms of metastatic cancer, administration of analgesics to control pain, management of constipation, and treatment of dyspnea and treatment of anemia, e.g., by transfusions, so as to maintain hemoglobin levels (i.e., ≥9 g/L). Other features of BSC for lung cancer are set forth below. In an embodiment according to the present invention, PSL docetaxel is administered in conjunction with a regimen of best supportive care. In another embodiment, PSL docetaxel can be the only chemotherapeutic anti-cancer agent administered to the patient. Preferably, the patient presents for PSL docetaxel treatment with Eastern Cooperative Oncology Group Performance Status (ECOG PS) of 0 or 1, and/or is less than 50 years of age. The patient may have stable disease or may have progressive disease at the time that PSL docetaxel therapy is begun.

The present method can further comprise administering an effective anti-emetic amount of a 5-HT3 receptor antagonist and dexamethasone to the patient.

In the embodiments provided herein, PSL docetaxel can be administered in doses spaced at about 3, 4, 5 or 6 week intervals, preferably at 3 week (21 day) intervals. In one embodiment of the invention, about 60 mg/m2-150 mg/m2, or in a second embodiment, preferably about 150 mg/m.sup.2 of picoplatin is administered in each dose. The dose may be administered orally or parenterally, or via combination of oral and parenteral routes. In one embodiment, the PSL docetaxel doses are administered by intravenous infusion of an aqueous solution of PLS docetaxel. The infusion of one dose is typically carried out over about one to two hours. Preferably, the solution is a physiological salt solution that has been previously adjusted to be isotonic with suitable salts. In one embodiment of the invention, about 0.5 mg/ml of docetaxel is present in the aqueous infusion solution, and contains at least one pharmaceutically acceptable tonicity adjuster, such as NaCl, MgCl₂, CaCl₂, KCl and the like. To achieve the preferred dosing, preferably about 200-300 mg of PSL docetaxel is administered per dose, e.g., per intravenous infusion.

Over the course of treatment of the cancer, 2-15 doses of PSL docetaxel can be administered, with 2-4 doses being typically administered, at intervals of about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, or more, (e.g., four to six weeks) or within a range of time defined by any two of the aforementioned time points.

In some embodiments, teach does provides between about 50-120 mg/m² docetaxel, and more preferably between about 75-90 mg/m² docetaxel. For example, in some embodiments, each dose provides 50 mg/m² docetaxel, 55 mg/m² docetaxel, 60 mg/m² docetaxel, 65 mg/m² docetaxel, 70 mg/m² docetaxel, 75 mg/m² docetaxel, 80 mg/m² docetaxel, 85 mg/m² docetaxel, 90 mg/m² docetaxel, 95 mg/m² docetaxel, 100 mg/m² docetaxel, 105 mg/m² docetaxel, 110 mg/m² docetaxel, 115 mg/m² docetaxel, 120 mg/m² docetaxel, or within a range defined by any two of the aforementioned amounts.

### Protein-Stabilized Liposomes

Provided herein are compositions and methods of administering protein-stabilized liposomes to subjects. Liposomes, have demonstrated biological properties that make them excellent pharmaceutical delivery systems, due in part to their biocompatibility, biodegradability, and low toxicity and are clinically used for efficacy enhancement and toxicity reduction. Liposomes have good longevity in the blood that allows their accumulation in pathological areas with compromised vasculature. Liposomes alter both the pattern of the distribution of therapeutics in the tissues and the rate of clearance by assigning the pharmacokinetic characteristics of the carrier to the therapeutic. The nanoparticle-sized liposomal vehicles as described herein are responsible for the enhanced permeability and retention effect (“EPR effect”). The EPR effect is a phenomenon whereby liposomal compositions having a size less than 500 nm (but not zero) tend preferentially accumulate in tissues (e.g., tumors), having defective vascular architecture, that enables the passage of nanoparticle-sized compositions through the “leaky” endothelium, rendering the particles particularly useful in the delivery of anti-neoplastic drugs, such as docetaxel.

When used in the delivery of certain cancer drugs, liposomes help to shield healthy cells from the drugs' toxicity and prevent their concentration in vulnerable tissues (e.g., the kidneys, and liver), lessening or eliminating the common side effects of nausea, fatigue, and hair loss.

Without wishing to be bound to any particular theory, the protein-stabilized liposomes described herein include protein molecules that minimize or eliminate drug leakage from the liposome. In accordance with the embodiments described herein, the protein-stabilized liposomes include a stabilizing protein. In certain embodiments, the stabilizing protein comprises an albumin (e.g., human serum albumin, or the like), immunoglobulin, casein, insulin, hemoglobin, lysozyme, immunoglobulin, α-2-macroglobulin, fibronectin, vitronectin, fibrinogen, lipase, and/or enzyme.

Many kinds of lipids may be used to create the protein-stabilized liposomes of the embodiments provided herein. For example, the liposomes described herein can be composed of naturally-derived phospholipids, including those with mixed lipid chains, with saturated phospholipids with long acyl chains (e.g., dipalmitoylphosphatidylcholine), or of pure surfactant components like DOPE (dioleoylphosphatidylethanolamine). Lipids that can be used to make the liposomes described herein include, but are not limited to, egg phosphatidylcholine (EPC), hydrogenated soy phosphatidylcholine (HSPC), phosphatidylethanolamine (PE), phosphatidylglycerol (PG), phosphatidylinsitol (PI), monosialogangolioside and spingomyelin (SPM), dipalmitoylphosphatidylcholine, dioleoylphosphatidylethanolamine, distearoylphosphatidylcholine (DSPC), dimyristoyl-phosphatidylcholine (DMPC), dimyristoylphosphatidylglycerol (DMPG), and dipalmitoylphosphatidylcholine (DPPC), and the like, or any combination thereof. Phospholipids are preferably used to create the protein-stabilized liposomes as described herein. In certain embodiments, the protein-stabilized liposomes include one or more of the following phospholipids: hydrogenated soy phosphatidylcholine (HSPC), egg phosphatidylcholine (EPC), phosphatidylethanolamine (PE), phosphatidylglycerol (PG), phosphatidylinsitol (PI), monosialogangolioside, spingomyelin (SPM), distearoylphosphatidylcholine (DSPC), dimyristoylphosphatidylcholine (DMPC), and/or dimyristoylphosphatidylglycerol (DMPG). In certain embodiments of the invention, the ratio of anti-neoplastic agent (e.g., docetaxel, or the like) to lipid-protein ranges from about 0.0005 to about 1 (w/w), more preferably about 0.0005 to about 0.5 (w/w), more preferably about 0.001 to about 0.1 (w/w) or within a range defined by any two of the aforementioned amounts. In certain embodiments of the invention, the ratio of anti-neoplastic agent (e.g., docetaxel, or the like) to lipid-protein ranges from 0.0005 to 1 (w/w), more preferably 0.0005 to 0.5 (w/w), more preferably 0.001 to 0.1 (w/w) or within a range defined by any two of the aforementioned amounts.

In some embodiments, the liposomes are polymer-coated liposomes. In some embodiments, the liposomes can include a polymer (preferably polyethylene glycol (PEG)), covalently conjugated to one of the phospholipids and provides a hydrophilic cloud outside the vesicle bilayer. This steric barrier delays the recognition by opsonins, allowing the liposomes to remain in circulation much longer than conventional liposomes (See, Lasic and Martin, Stealth Liposomes, CRC Press, Inc., Boca Raton, Fla. (1995)). Accordingly, in some embodiments, the protein-stabilized liposomes described herein can include a PEG-phospholipid. For example, in certain embodiments of the invention, the protein-stabilized liposome can include poly(ethylene glycol)-derivatized distearoylphosphatidylethanolamine (PEG-DSPE) and/or poly(ethylene glycol)-derivatized ceramides (PEG-CER). The PEG-phospholipid and antineoplastic agent (e.g., docetaxel) may be included in an organic solution at a ratio ranging from about 0.01:25 to about 1:10 drug:lipid (w/w). Polymer-derivatized lipids and/or phospholipids comprised of methoxy(polyethylene glycol) (mPEG) and a phosphatidylethanolamine can be used in the embodiments disclosed herein. Such derivatized phospholipids (e.g., dimyristoyl phosphatidylethanolamine, dipalmitoyl phosphatidylethanolamine, distearoyl phosphatidylethanolamine (DSPE), or dioleoyl phosphatidylethanolamine) can be obtained from Avanti Polar Lipids, Inc. (Alabaster, Ala.) at various mPEG molecular weights (350, 550, 750, 1000, 2000, 3000, 5000 Daltons). Polymers of mPEG-ceramide can also be purchased from Avanti Polar Lipids, Inc. Preparation of lipid-polymer conjugates useful in the embodiments disclosed herein is also described in the literature, see U.S. Pat. Nos. 5,631,018, 6,586,001, and 5,013,556 (all incorporated by reference); Zalipsky, S., et al., Bioconjugate Chem. 8:111 (1997); Zalipsky, S., et al., Meth. Enzymol. 387:50, (2004). In some embodiments, the polymer-derivatized lipids can also be a polymer-distearoyl conjugate, as described in U.S. Pat. No. 6,586,001, incorporated by reference herein.

In addition to phospholipids, in preferred embodiments, the protein-stabilized liposomes provided herein include cholesterol.

In some embodiments, the liposomes can include a mixture of egg phosphatidylcholine (EPC), hydrogenated soy phosphatidylcholine (HSPC), phosphatidylglycerol (PG), phosphatidylinositol (PI), monosialoganglioside and sphingomyelin (SPM); the derivatized vesicle forming lipids such as poly(ethylene glycol)-derivatized distearoylphosphatidylethanolamine (PEG-DSPE), poly(ethylene glycol)-derivatized ceramides (PEG-CER), di stearoylphosphatidylcholine (DSPC), dimyristoyl-phosphatidylcholine (DMPC), dimyristoylphosphatidylglycerol (DMPG), and/or dipalmitoylphosphatidylcholine (DPPC), cholesterol, and/or proteins.

Sugars, sugar alcohols, and other compounds are known to promote stabilization of liposomal compositions. Accordingly, in some embodiments, the liposomes disclosed herein include, for example, monosaccharides such as glucose, galactose, mannose, fructose, inositol, ribose, and/or xylose; disaccharides such as lactose, sucrose, cellobiose, trehalose, and/or maltose; trisaccharides such as raffinose and/or melizitose; polysaccharides such as cyclodextrine; and/or sugar alcohols such as erythritol, xylitol, sorbitol, mannitol, and/or maltitol. In some embodiments, the liposomes described herein can include blends of glucose, lactose, sucrose, trehalose, and/or sorbitol, or blends of lactose, sucrose, and/or trehalose, or the like. By this approach, the liposomal compositions can be stably stored over long periods.

In embodiments where the liposomes are frozen, the liposomes preferably contain polyvalent alcohols (aqueous solutions) such as glycerin, diglycerin, polyglycerin, propylene glycol, polypropylene glycol, ethylene glycol, diethylene glycol, triethylene glycol, polyethylene glycol, ethylene glycol monoalkylether, diethylene glycol monoalkylether and/or 1,3-butylene glycol. With respect to polyvalent alcohols (aqueous solutions), glycerin, propylene glycol, polyethylene glycol are preferable, and glycerin and propylene glycol are more preferable. By this means, it is possible to stably store the liposomes over long periods. In some embodiments, the liposomes include both sugars and polyvalent alcohols in combination.

Preferably, the protein-stabilized liposomes described herein have an average diameter less than 500 nm (but not zero). In some embodiments, the average diameter of the liposomes described herein is less than (but not zero) about 500 nm, less than about 450 nm, less than about 400 nm, less than about 350 nm, less than about 300 nm, less than about 250 nm, less than about 200 nm, less than about 150 nm, less than about 100 nm, less than about 90 nm, les than about 80 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm, or of a size within a range defined by any two of the aforementioned sizes. In some embodiments, the average diameter of the liposomes described herein is less than (but not zero) 500 nm, less than 450 nm, less than 400 nm, less than 350 nm, less than 300 nm, less than 250 nm, less than 200 nm, less than 150 nm, less than 100 nm, less than 90 nm, less than 80 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, or of a size within a range defined by any two of the aforementioned sizes. In some embodiments, the compositions disclosed herein comprise liposomes that have a cross-sectional diameter of no greater than about 10 microns or 10 microns. A cross-sectional diameter of less than 5 microns (but not zero) is more preferred, while a cross-sectional diameter of less than 1 micron (but not zero) is presently the most preferred.

In preferred embodiments, the protein-stabilized liposomes described herein include, for example, DSPC, PEG-DSPC, cholesterol, docetaxel, human serum albumin, and sucrose.

### EXAMPLES

### Example 1: Pharmacokinetics of Protein-Stabilized Liposomal Docetaxel Versus Free Docetaxel

The following experiments were performed to assess the pharmacokinetic profile of docetaxel administered in a protein-stabilized liposome in a Yucatan female minipig.

PSL docetaxel was prepared according to the methods set forth in U.S. Pat. No. 7179484. On Day 1, 11.9 mg of Docetaxel was dissolved in 1.2 ml of absolute ethanol, mixed with 3.0 ml polysorbate tween 80 and added to a final volume of 11.9 ml with 0.9% saline to make a stock concentration of 1.0 mg/ml docetaxel. The animal was infused with 11.90 ml of the solution at a rate of 0.5 ml per minute. The administration volume was determined by calculating the dose (0.75 mg/kg) of compound adjusted to the weight of animal (15.8 kg) 24 hours prior to start date of study. PSL docetaxel was provided at a concentration is 1 mg/ml.

Docetaxel was dosed either alone (free docetaxel) or formulated as PSL docetaxel in one naive Yucatan minipig with an eleven day washout period. Plasma samples were taken at pre-determined time points following dosing over 72 hours.

Blood samples were collected into K2EDTA tubes from animal dosed in prior to dosing; 5, 15, and 30 minutes after dosing; and 1, 2, 4, 8, 24, 36, 48, and 72 hours after dosing. The time of sample collection were recorded. At each time point, 1 ml of blood was collected into K2EDTA tubes and processed to plasma by centrifuging at approximately 2,000 rpm for approximately 10 minutes. The cellular fraction of the blood was discarded. Samples were stored at -80 C until used for analytic analysis.

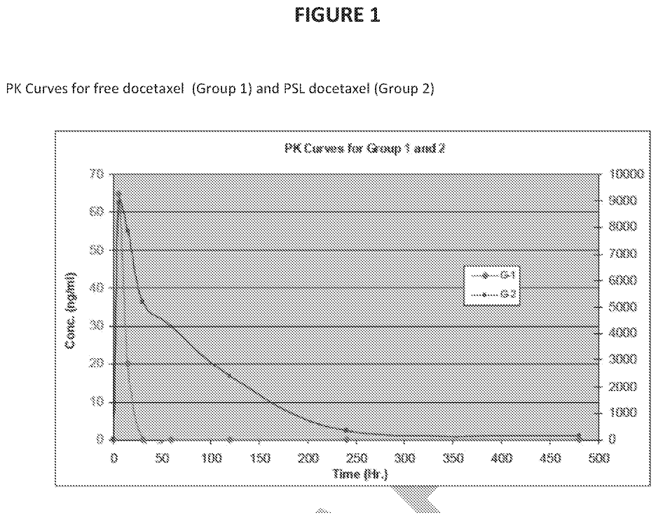

The PK curves for the free docetaxel and PSL docetaxel are shown in FIG. 1. The data demonstrated that there are marked differences in docetaxel plasma kinetics following administration of PSL docetaxel as compared to free docetaxel. As shown in FIG. 1, plasma concentrations of total docetaxel were consistently higher for PSL docetaxel compared to free docetaxel over the entire exposure period.

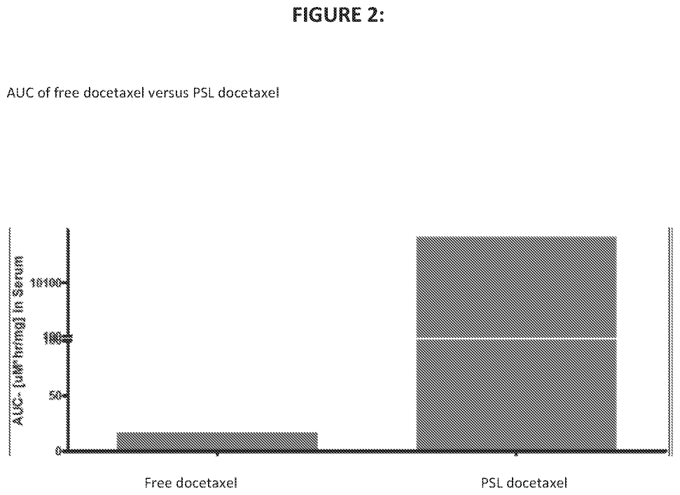

As shown in FIG. 2, PSL docetaxel had an AUC value that was greater than 1,000× that seen for free docetaxel.

These data suggest that PSL docetaxel will have a superior therapeutic effect as compared to free docetaxel.

### Example 2: Biodistribution of Ati-1123 Compared to Free Docetaxel

The following experiments were performed to analyze the biodistribution of PSL docetaxel, as compared to free docetaxel.

PSL docetaxel was prepared as described herein. Three groups of mice were used in the study, each containing 5 or 25 mice (see below). Mice were implanted with tumor fragments of PC-3 tumors from nude mice hosts. When tumors grew to approximately 100 mm³ in size, animals were randomized into treatment and control groups. The treatment groups were as shown in Table 2.

TABLE 2

| Group | # Animals | Compound | Dose (mg/kg) | Route/Schedule |
|---|---|---|---|---|
| 1 | 5 | No treatment | — | — |
| 2 | 25 | Free docetaxel | 25 | IV/QDx1 |
| 3 | 25 | PSL docetaxel | 25 | IV/QDx1 |

Tumor, plasma and tissue samples including lungs, liver, kidneys, pancreas, heart, and brain were collected from treated animals at 1, 4, 8, 24 and 48 hours post-dose by destructive sampling technique (5 mice per time point). Samples from four untreated animals were also collected on Day 1 as controls. All samples were weighed, flash frozen in liquid nitrogen, and stored at −80° C. until analyzed.

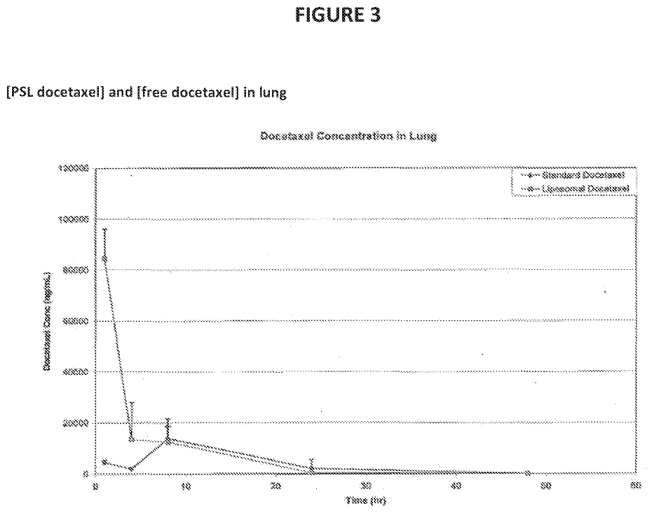

Plasma concentrations were higher following dosing with PSL docetaxel compared with those seen after dosing with free docetaxel. Surprisingly, amount of docetaxel was detected in the lungs of mice that had received ATI-1123 when compared to the mice that had received free docetaxel, during the first four hours after dosing. By eight hours post-dosing, the docetaxel accumulation in the lungs was the same between the PSL docetaxel and free docetaxel groups (see, FIG. 3).

The foregoing data demonstrate that the PSL docetaxel formulation of docetaxel substantially alters the tissue distribution, and suggest that PSL docetaxel will have a superior therapeutic effect in treatment of lung cancers, including but not limited to SCLC, when compared to free docetaxel.

### Example 3: Pharmacokinetic and Safety of Ati-1123 in Humans

This example describes a phase I clinical trial that assessed the safety and pharmacokinetics of various doses of PSL docetaxel in humans.

Criteria for enrollment were that the subject was that had histologically confirmed measurable solid tumor and that had progressed following standard/approved chemotherapy or had no appropriate alternative therapy available; had an Eastern Cooperative Oncology Group (ECOG) performance status of 0-1 and life expectancy ≥3 months. Subjects with NYHA Class III or IV cardiac disease, myocardial infarction within the last 6 months unstable arrhythmia, or evidence of ischemia on echocardiogram were excluded. Also excluded were patients that had active CNS disease or active, uncontrolled bacterial, viral, or fungal infections requiring systemic therapy or seizure disorders requiring anticonvulsant therapy; had severe, chronic obstructive pulmonary disease with hypoxemia; were pregnant or lactating; or exhibited allergic reactions to docetaxel, or a similar structural compound, biological agent, or formulation.

PSL docetaxel was administered as an intravenous infusion every three weeks at the following doses: 15 mg/m², 30 mg/m², 60 mg/m², 75 mg/m², 90 mg/m², or 110 mg/m².

### Pharmacokinetics:

The pharmacokinetic (PK) endpoints were the PK parameters calculated from the PSL docetaxel plasma concentrations obtained prior to initiating the infusion of PSL docetaxel on Day 1, at the completion of the infusion, and post-infusion at 15 and 30 minutes and at 1, 2, 4, 8-10, 24, and 48 hours after completion of the infusion. On Day 8, a blood sample for PK analysis was also collected. In addition, blood samples were collected in Cycle 2 from the patients at the maximum tolerated dose who received repeated doses of PSL docetaxel at predose and end of infusion. PK endpoints included: Cmax , AUC0-t , AUC0-∞, t½, and Vd.

All samples were assayed for total plasma docetaxel concentrations. The plasma ultrafiltrate of the samples was also assayed for free docetaxel and the concentration of bound docetaxel was calculated. Quantification of docetaxel concentrations were performed using validated LC-MS/MS methods.

All of the analyses to be performed on the PK plasma concentrations of ATI 1123 obtained from patients in the PK Population were performed separately for three analytes: (i) concentrations of free ATI-1123; (ii) (calculated) concentrations of bound ATI 1123; and (iii) total concentrations of ATI-1123. Summaries of the ATI-1123 plasma concentrations at each measurement time point were prepared. Additionally, a comparison of the ATI-1123 plasma concentrations following the first dose (given on Day 1) was compared to those following the second dose (Day 22; Cycle 2 Day 1) using paired t-tests. Pharmacokinetic parameters based on the plasma concentrations of ATI-1123 obtained during the first cycle were computed using non-compartmental methods. These calculations were performed using SAS® (release 9.1 or higher), or using validated equivalent pharmacokinetic software programs from other vendors. The pharmacokinetic parameters calculated included: Cₘₐₓ, AUC₀₋ₜ, AUC_{0-∞}, t½ Additionally, the dose proportionality of ATI-1123 AUC_{0-∞}. Summaries of these parameters were provided. Additionally, the dose proportionality of ATI-1123 of AUC_{0-∞} and Cₘₐₓ was evaluated through the preparation of figures, and the calculation of correlation coefficients.

### Pharmacodynamics:

α1-cid glycoprotein (AAG) concentrations were correlated to the PK data, toxicity, and activity of ATI-1123. If the patient had a known history of PET-positive tumor(s) based on prior exams, and following agreement with the Medical Monitor and Investigator, then PET scans were permitted to evaluate response.

### Anti-Neoplastic Activity:

Analyses investigating antitumor activity were performed using data from the PD Population. For each evaluation time point, the antitumor effects of ATI-1123 were estimated from the numbers and percentage of patients with complete and partial responses, stable disease, and progressive disease following ATI-1123 treatment using RECIST v1.1.

All patients with progressive disease reported at any time during the study were included in a listing. The data for tumor assessments were presented in a listing.

### Safety:

All safety analyses were performed on the Safety Population. Safety observations and measurements included drug exposure, AEs, laboratory tests, vital signs, body weight, physical examinations, ECGs, and ECOG performance status. Treatment-emergent AEs were coded by body system and preferred term using the Medical Dictionary for Regulatory Activities (MedDRA) Dictionary. AEs were tabulated by highest toxicity grade and relationship to PSL-docetaxel. Serious AEs (SAEs), deaths, AEs resulting in discontinuation, dose reductions, and dose interruptions were listed. The number and percentage of all AEs and SAEs collected during the two required cycles were summarized at each dose level for each cycle. All adverse events, including those collected beyond the first two cycles, were listed. Dose-limiting toxicities reported during this study were provided in a listing.

### Results:

PSL docetaxel, at the doses tested, had an acceptable safety and tolerability profile when administered as an IV infusion on Day 1 every 3 weeks to patients with advanced solid malignancies. The maximum tolerated dose was determined to be 90 mg/m². Pharmacokinetic analysis revealed an apparent enhanced exposure to docetaxel in patients treated with ATI-1123 when compared to the exposure levels observed in patients treated historically with standard docetaxel.

The PSL formulation of docetaxel enhanced the exposure of docetaxel in human subjects. Encapsulated docetaxel plasma concentrations were generally greater than the nonencapsulated docetaxel concentrations (approximately a 4-fold increase). The Cmax and AUC(0-inf) values for encapsulated, non-encapsulated and total docetaxel were all proportional to ATI-1123 dose.

No patient had a complete response. One patient's tumor shrank by 61% and the PR lasted for approximately 26 weeks. Twenty-two patients had a best response of stable disease with tumor shrinkage ranging from 1% to 29% and the duration of stable disease ranging from 6 weeks to 57 weeks.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of inhibiting, a small cell lung cancer in a subject, comprising:
identifying a subject with small cell lung cancer; and
administering to the subject a therapeutically effective amount of a protein-stabilized liposome that comprises docetaxel..

2-41. (canceled)

42. The method of claim 1, wherein the subject has refractory SCLC.

43. The method of claim 1, wherein the subject was non-responsive to an initial therapy.

44. The method of claim 1, wherein the subject responded to an initial therapy but relapsed within 3 months of cessation of said initial therapy.

45. The method of claim 1, wherein the subject has sensitive SCLC.

46. The method of claim 44, wherein the subject's response to initial therapy was a complete response.

47. The method of claim 44, wherein the subject's response to initial therapy was a partial response.

48. The method of claim 1, wherein the subject has limited-stage SCLC.

49. The method of claim 1, wherein the subject has extensive-stage SCLC.

50. The method of claim 1, wherein the therapeutically effective amount is sufficient to extend progression-free survival of the subject.

51. The method of claim 1, wherein the therapeutically effective amount is sufficient to extend overall life expectancy of the subject.

52. The method of claim 1, wherein the therapeutically effective amount is between 50-120 mg/m² docetaxel.

53. The method of claim 1, wherein the therapeutically effective is between 75-90 mg/m² docetaxel.

54. The method of claim 43, wherein the initial therapy comprises a platinum-containing anti-cancer agent.

55. The method of claim 1, wherein the protein-stabilized liposome comprises albumin, an albumin derivative or an albumin mutant.

56. The method of claim 1, wherein the protein-stabilized liposome comprises methoxypolyethylene glycol (MPEG).

57. The method of claim 1, wherein the subject has refractory SCLC and the overall survival of said subject is improved.

58. The method of claim 1, wherein the subject has refractory SCLC and the progression-free survival is improved.

59. The method of claim 56, wherein the MPEG has a molecular weight of 350, 550, 750, 1000, 2000, 3000, or 5000 Daltons.
